(19) United States
(12) Patent Application Publication
(10) Pub. No.: US 2017/0360476 A1
(43) Pub. Date: Dec. 21, 2017

(54) TROCAR FOR IMPROVED FLUID PRESSURE MANAGEMENT DURING ENDOSCOPIC SURGERY

(71) Applicant: University of Louisville Research Foundation, Inc., Louisville, KY (US)

(72) Inventor: George M. Pantalos, Louisville, KY (US)

(73) Assignee: University of Louisville Research Foundation, Inc., Louisville, KY (US)

(21) Appl. No.: 15/524,115

(22) PCT Filed: Dec. 1, 2015
(86) PCT No.: PCT/US15/63125

§ 371 Date: May 3, 2017

Related U.S. Application Data
(60) Provisional application No. 62/085,771, filed on Dec. 1, 2014.

(51) Int. Cl. A61B 17/34

(52) U.S. Cl. CPC A61B 17/3462; A61B 17/3498

(57) ABSTRACT

A trocar for arthroscopy and other endoscopic surgeries is provided, including a diaphragm seal, in which the assembly as a whole provides adequate insufflation of the body compartment during a procedure, while limiting or eliminating the leakage of insufflation fluid from the rear of the trocar, thus maintaining adequate insufflation pressure management. The design, placement, and spacing associated with said diaphragm seal provides an enhanced interface between the diaphragm opening and a surgical instrument inserted through such opening to gain access to the body compartment of a surgical patient.

### PRIORITY STATEMENT

This international patent application claims benefit under the Paris Convention to U.S. Provisional Patent Application Ser. No. 62/085,771, with a filing date of 1 Dec. 2014.

### FIELD OF INVENTION

Embodiments described herein relate to a trocar for arthroscopic, laparoscopic, and other endoscopic surgeries, marked by an improved seal interface between a surgical instrument and the trocar, thus promoting visual clarity and improved fluid pressure management for maintaining adequate distention of tissues within a body compartment of a surgical patient.

### BACKGROUND

Endoscopic surgery refers to surgical procedures performed through small, puncture-like incisions which serve as an entry point(s) to the surgical site. The incisions are made through skin and subcutaneous tissues using a trocar. One end of a trocar has a tip that is appropriately sized for insertion through a small incision to access the surgical site. A surgeon can then pass other surgical instruments through the trocar to conduct the surgery. Examples of such instruments include scalpels, graspers, scissors, staplers, and scopes, to name a few. Various forms of endoscopic surgery include, without limitation, arthroscopy, laparoscopy, thoracoscopy, cystoscopy, and microsurgery, to name some. In some surgical disciplines (e.g. orthopedic surgery), the term “cannula” is used synonymously with the term “trocar.”

Arthroscopic surgery, or arthroscopy, is a type of endoscopic surgery that deals with bones, joints, and connective tissues. It is estimated there are more than 1 million arthroscopies in the U.S. annually involving the knee alone, and studies have indicated a growth rate over the last decade approaching 50%. Visual clarity of the surgical field is an important consideration for endoscopic surgeries. Conventionally, this objective is furthered by a process known as insufflation, which injects a fluid into the body compartment to distend the tissues within or surrounding a body compartment associated with a surgical site. For example, gas is often injected into the body compartment to distend the tissues for laparoscopic surgery, liquid is often used in arthroscopic surgery, and sometimes, depending on the nature of the operation, vapors or powders are used. Often, injection is through a port that can be opened or closed, such as by a stopcock or like structures, located between the trocar's insertion tip and a valve generally positioned proximal to a forward insert region of the trocar.

However, adequate insufflation requires a steady pressure within the body compartment, or else adequate distention may not be maintained. But considering that the insufflation fluids are injected through a port in the trocar, if the trocar itself is not properly sealed, then leakage of insufflation fluid is likely to occur, and there may not be enough pressure to maintain adequate distention. Conversely, compensating for leakage by over-pressurization might lead to complications and increased risk of adverse events for the patient. While most trocars provide a single diaphragm seal for the purpose of sealing off the external environment, these conventional designs are known to permit fluid to leak out of the end of the trocar—often with the fluid traveling along the tip of the instrument. Such leakage reduces the pressure in the body compartment and allows fluid to leak into the external surgical field. This can occur during insertion and/or withdrawal of an instrument, or when no instrument is positioned within the trocar. Excessive leakage results in poor pressure management within the body compartment, wastes insufflation fluid, and may create a safety hazard for operating room personnel.

As previously mentioned, a trocar is used to pass endoscopic surgical instruments into the body compartment of a patient. Conventionally, a surgical instrument is inserted through the trocar, generally through an opening at the rear aspect (i.e., distal to the entry point of the trocar tip as it enters the patient's body compartment). The instrument is then advanced toward the surgical site. Such instruments (i.e., surgical instruments) generally have a tip (i.e., an operational end such as, for example, scissor/cutting members and graspers), a shaft, and a handle. Guided by the surgeon, the tip of the instrument travels from a rear insert of the trocar (proximal to the surgeon) through the body of the trocar until exiting the trocar at a forward insert (distal to the surgeon and positioned within a body compartment of a patient). With conventional designs, as the tip of the instrument enters the trocar, it first passes through a single, flat (i.e., not tapered) diaphragm seal which is positioned proximal to the rear insert and generally within the inner chamber of the trocar body. The instrument tip then passes through a valve within the chamber of the trocar, wherein this valve is proximal to the forward insert. Conventional trocar designs position the diaphragm seal and valve in relatively close proximity.

A surgical instrument, having a tip, is advanced through the interior of the trocar during operation. Problematically, if the length of the instrument tip exceeds the distance between a conventional outer diaphragm seal and the interior valve, the valve opens before the instrument shaft fully engages the outer seal. The resulting inadequate seal produces leakage as insufflation fluid, under the pressure needed for injection through the port, passes along the instrument tip. This frequently causes fluid to escape from the trocar housing along the shaft of the instrument, because the partial engagement between the shaft and the diaphragm seal prevents an adequate seal against the external field.

The present embodiments herein alleviate this problem by enabling full sealing engagement to occur between the instrument shaft and a diaphragm seal, before the tip of the instrument engages the forward-most valve while being advanced after insertion, for example.

### SUMMARY OF EMBODIMENTS

A trocar, according to multiple embodiments and alternatives as provided herein, comprises a diaphragm seal having two opposing tapered portions, at least one of which forms a sealing surface that is tapered, as opposed to flat. Such a dual-taper diaphragm seal arrangement, relative to positioning of valves within the trocar itself, offers the benefits of virtual leak-free performance of the trocar during endoscopic surgery. In some embodiments, a diaphragm seal and one or more valves of the subject trocar are configured such that the diaphragm seal is positioned exterior of the trocar inner chamber, with one or more interior valves positioned inside the trocar inner chamber. The effect is to increase the opening-to-opening distance between the diaphragm seal and the forward-most valve, the latter of which provides access to the surgical site. The added distance prevents insufflation fluid from leaking out prior to the time when the seal is fully formed between the instrument and such diaphragm seal. In some embodiments, the valves are multi-leaflet valves.

The arrangement provided herein allows for sufficient thickness at the diaphragm seal opening for continuous contact with the instrument to maintain a fluid tight, mechanical seal between the shaft of a surgical instrument and the diaphragm seal. Moreover, this fluid tight seal withstands pressures at and beyond what is typically exerted by means of insufflation, and the forward-facing tapered portion is configured to redirect insufflation fluid into the interior of the trocar chamber and away from the diaphragm seal opening.

In some embodiments, the novel diaphragm seal is secured to a seat region of the trocar by means of an end cap, which matably threads to corresponding threads on the housing of the trocar. In some embodiments, multiple diaphragm seals are used, such that a first diaphragm seal is located within the inner chamber of the trocar housing, while a second diaphragm seal can be located either just beyond the rear insert of the trocar housing, or externally through means of the aforementioned end cap. The use of one or more diaphragm seals having a dual taper along with the arrangement of valve(s) and diaphragm seal(s) according to present embodiments provides virtually leak-free handling during the insertion, use, and withdrawal of surgical instruments associated with endoscopic surgeries.

The shaft of many surgical instruments has a round cross-section axially, generally of a standard diameter depending on several factors, including but not limited to the type of instrument, the nature of the procedure, and the size of the body compartment and patient. According to multiple embodiments and alternatives herein, the design of a trocar, with one or more diaphragm seals, can be scaled and tailored to accommodate use of various instruments based on factors such as those mentioned above. In some embodiments, a diaphragm seal according to these descriptions can be fitted to a conventionally available trocar. Among various options, such a diaphragm seal is included with a new trocar, either being formed integrally with the trocar; or provided as a separate piece that can be fitted over an end cap or seat region of a trocar; or incorporated with a trocar internally. Other features and advantages will be apparent based on the descriptions herein.

### BRIEF DESCRIPTION OF THE FIGURES

The drawings, schematics, figures, and descriptions herein are to be understood as illustrative of structures, features and aspects of the present embodiments and do not limit the scope of the embodiments. The scope of the application is not limited to the precise arrangements, scales, or dimensions as shown in the drawings, nor as discussed in the textual descriptions.

FIG. 1 is a perspective view of a trocar, according to multiple embodiments and alternatives described herein.

FIGS. 2 and 3 are cross-sectional views of a trocar with an instrument positioned as it would be in use, taken along line II-II of FIG. 1, according to multiple embodiments and alternatives described herein.

FIGS. 4A-4C provide cross-sectional views of the diaphragm seal portion of region “C” denoted in FIG. 3, but without picturing the instrument shaft 5, illustrating several alternatives according to the embodiments herein.

FIGS. 5A and 5B offer a perspective view of a diaphragm seal for a trocar, according to multiple embodiments and alternatives described herein.

FIGS. 6A and 6B offer a perspective view of a diaphragm seal situated on a trocar according to multiple embodiments and alternatives, in which the diaphragm seal is operatively engaged with an instrument shaft.

FIG. 7 is a cross-sectional view taken lengthwise of an endcap for a trocar such as of the end cap portion along line II-II of FIG. 1, having a seat arranged to accommodate a diaphragm seal, according to multiple embodiments and alternatives described herein.

FIG. 8 is a cross-sectional view taken lengthwise of a trocar housing with grooves formed in the inner wall surface, taken along line II-II of FIG. 1, according to multiple embodiments and alternatives described herein.

The surgical instrument shown in some figures, including the tip of the instrument and the shaft, is not part of the claimed embodiments herein.

### MULTIPLE EMBODIMENTS AND ALTERNATIVES

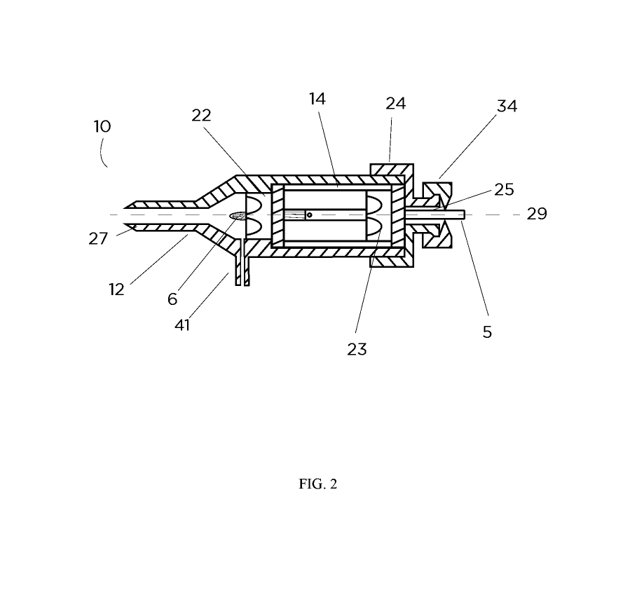

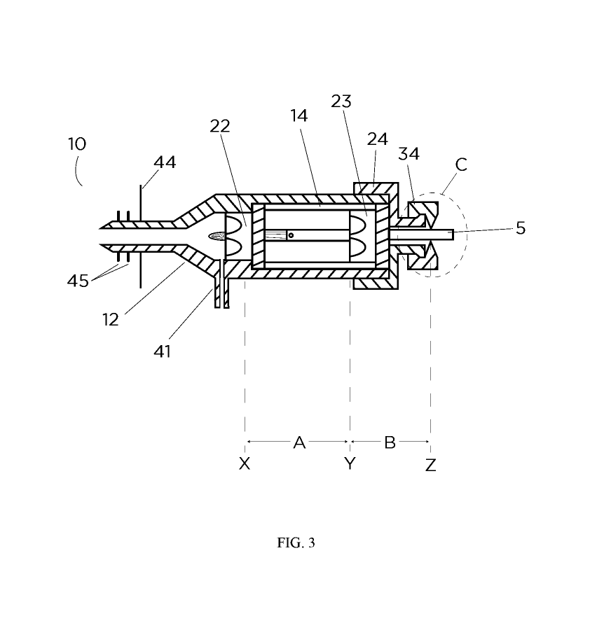

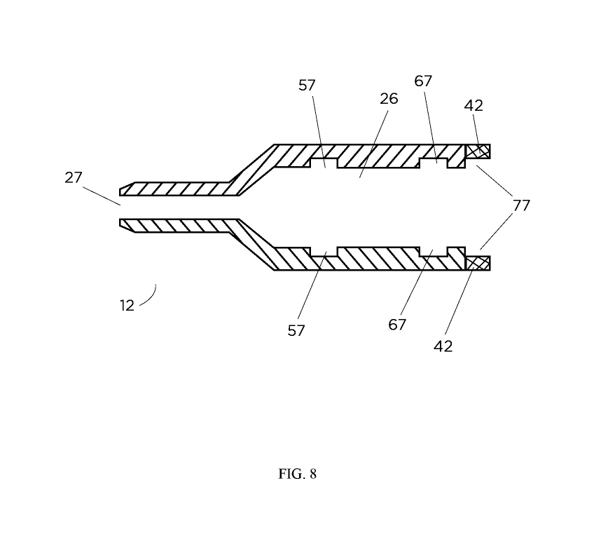

Present embodiments concerning a trocar 10 relate to a number of standard parts related to conventionally designed trocar devices, for use with conventional surgical instruments. For example, FIG. 2 shows the trocar in use with an instrument having a tip 6 (which may be hinged), an instrument shaft 5, and a handle (not shown). Other elements commonly found in conventional trocar designs, some of which are optional, are shown in various figures, such as FIG. 3 and FIG. 8. These include a cylindrical housing 12, inner chamber 26, at least one valve 22, rear insert 25, forward insert 27, skin interface seal 44, and one or more spiral skin threads 45. Typically, the skin interface seal 44 and spiral skin threads 45 are involved in gaining access to the body compartment through the patient's skin. Accordingly, the skin interface seal 44 and spiral skin threads 45 generally are positioned proximally relative to the forward insert 27. By the teachings herein, these structures and the forward insert are oriented “forward” with respect to trocar 10, while the rear insert 25 is oriented “rear” (or, “rear-ward”) with respect to trocar 10.

The main function of trocar 10 is to provide access to the body compartment. The shaft 5 of most surgical instruments used for endoscopy has a round cross-section of a standard diameter depending on various factors, e.g., the type of instrument, the nature of the procedure, and the size of the body compartment and patient. The diaphragm seal of trocar 10 must sufficiently engage with an instrument shaft to establish a fluid tight seal along the abutting surface of the instrument shaft. As used herein, the term “fluid tight seal” indicates that the seal is substantially impermeable to fluids under pressures at and beyond that which is typically exerted by means of insufflation, as described in more specificity below. In addition, the term “fluid,” as used herein, is intended to include gases, such as air, nitrogen, carbon dioxide and the like, liquids, such as water or saline, and/or any matter, substance or combination of compounds substantially not in a solid state, or in an otherwise effectively immobile condensed state.

The novel diaphragm seal works in conjunction with one or more valves that are positioned within inner chamber 26 of trocar 10. Conventionally, valves 22, 23 as depicted in FIGS. 2 and 3 are formed from polymer materials as are known in the art, and positioned within inner chamber 26 in order to allow portions of a surgical instrument including the tip 6 to pass through their respective openings (during advancement and withdrawal), while also acting as a seal to prevent fluid passage when an instrument has not been passed through that valve. FIGS. 2 and 3 also show diaphragm seal 34 arranged with end cap 24, as further discussed below in connection with other figures. In some embodiments, the trocar is provided by itself, while optionally a trocar as described herein is combined with particular endoscopic surgical instruments, and intended for single and/or multi-use purposes.

Suitable materials for trocar 10, end cap 24, and diaphragm seal 34 are known in the art. By way of non-limiting example, suitable materials for trocar 10 include polycarbonate acrylic, plastics, and metals such as titanium alloys and steel alloys; suitable materials for end cap 24 include plastics and metals; and suitable materials for diaphragm seal 34 include silicone elastomers and rubbers, to name a few. In an embodiment, the diaphragm seal 34 is formed from a silicone rubber compound that is commercially sold as Dragon Skin® High Performance Silicone Rubber, and available from Smooth-On, Inc., of Macungie, Pa. As desired, and consistent with various embodiments and alternatives, the aforementioned materials can be machined, molded, injection molded, additively manufactured, or otherwise formed through techniques which are well known in the art.

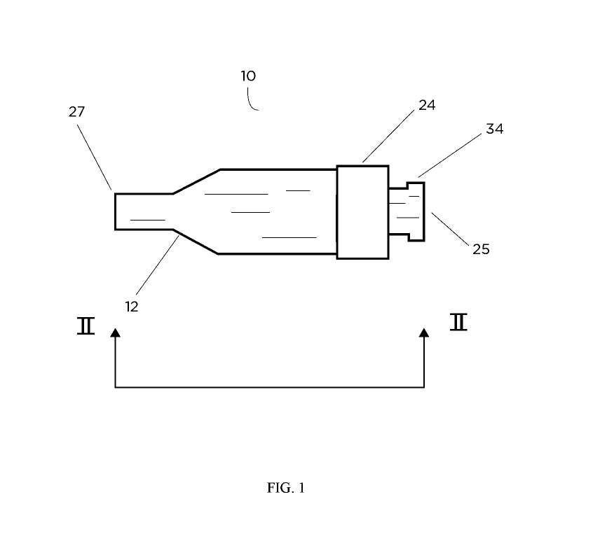

As previously referenced, FIG. 2 is a cross-sectional view of a trocar, taken axially along line II-II of FIG. 1. In some embodiments, trocar 10 comprises two valves (22, 23) with a diaphragm seal 34 as described herein arranged with end cap 24, external to the trocar housing 12. Alternatively, with reference to FIG. 8, diaphragm seal 34 is positioned internally, for example by forming a press fit within end cap 24, or by seating outer edge 31′ of the diaphragm seal securely within a grooved portion 77 of the housing, or otherwise positioning it within trocar housing 12, generally to the rear of the valves 22, 23.

Generally, end cap 24 is at least partially open in its rear-facing orientation to accommodate passage of the tip and shaft of the instrument while advancing through (or being withdrawn axially from) trocar 10. In some embodiments, valves 22, 23 are multi-leaflet valves. Accordingly, in the drawings a first valve 22 is referred to synonymously as “forward valve,” and a second valve 23 is referred to as “rear valve.” In both FIGS. 2 and 3, an optional spacer sleeve 14 is shown, which can be made from a number of optional materials, including of the same materials which are used in forming the trocar itself. In some embodiments, spacer sleeve 14 is a cylindrical sheath with a hollow central area so as not to impede the passage of instruments through inner chamber 26, and it fits within inner chamber 26 snugly within trocar housing 12. In some embodiments, the geometry of spacer sleeve 14 is complementary to that of the interior of trocar housing 12. By its placement, spacer sleeve 14 urges first valve 22 towards a forward insert 27 along axis 29 in relation to trocar 10, which generally follows the path of advancement of a surgical instrument during use, for example as the instrument is advanced and later withdrawn between the rear insert 25 and the forward insert 27.

Present embodiments contemplate various alternatives for maintaining suitable distances between valves 22 and 23 and the diaphragm seal 34, respectively. For example, FIG. 3 shows spacer sleeve 14 for maintaining suitable spacing and distances between the opening 35 of diaphragm seal 34, and the respective openings of valves 22, 23. Preferably, during use the distance between these valves is substantially statically maintained. In addition to a spacer sleeve, other structures can be employed to maintain suitable spacing and distances. In some embodiments, valves 22, 23 are positioned within the inner chamber 26 by forming grooves 57, 67 in the inner wall of trocar housing 12, as shown in FIG. 8. In such embodiments, the circumferential ring of valve 22 seats securely in groove 57 of the housing; likewise, the circumferential ring of valve 23 seats securely in the grooves 67. Accordingly, the respective grooves each provides a valve seat region to position valves 22, 23 relative to the other and to diaphragm seal 34. The distance between these grooves determines the spacing between openings for valve 22, 23 and by extension the distance between each of those valves and diaphragm seal 34. The fit between the valves and the grooves is configured to hold each valve in a substantially static position within chamber 26.

FIG. 3 is a cross-sectional view of a trocar, showing positioning of a port 41, which is a conventional port for trocars to provide means for passing insufflation fluids such as carbon dioxide. FIG. 3 also illustrates, in non-limiting fashion, some spacing aspects which are suitable for certain embodiments and for certain uses based on the type of patient, for example as a function of size differences between adult patients and pediatric patients. In some embodiments, the spacing is sufficient to allow, as the instrument shaft advances axially, a fluid tight seal to form at opening 35 before the tip of the surgical instrument passes through the opening of rear valve 23. Likewise, upon withdrawal of the instrument axially in a generally rear-ward direction, it is beneficial if a fluid tight seal remains at opening 35 as the tip of the surgical instrument exits the opening of forward valve 22. Accordingly, in some embodiments, the distance from the diaphragm seal opening 35 to the opening of rear valve 23 is distance “B”, and represented by points “Y” to “Z”. Likewise, the distance from diaphragm seal opening 35 to the opening of forward valve 22 is the sum of distances “A” and “B”, and represented by points “X” to “Z.”

In view of the above, in some embodiments, a distance from position X to position Y represents the spacing between the opening of valve 22 and the opening of valve 23 (denoted as distance A in FIG. 3). In some embodiments, this distance A measures from about 1 centimeter (cm) to about 2 cm for pediatric applications, and from about 4 cm to about 5 cm for adult applications. Similarly, a distance from point Y to point Z (denoted as distance B in FIG. 3) represents the spacing between the opening of valve 23 and an opening 35 of diaphragm seal 34, with this distance measuring from about 1 cm to about 2 cm for pediatric applications, and from about 4 cm to about 6 cm for adult applications. Accordingly, in some embodiments, the distance from the diaphragm seal opening 35 to the opening of rear valve 23 is at least 2 cm, and the distance from this diaphragm seal opening to the opening of forward valve 22 is at least 7 cm. Thus, while advancing an instrument, the spacing as described herein allows for the instrument shaft to form a fluid tight seal with diaphragm seal 34 before the instrument tip meets the opening for rear valve 23. Likewise, during withdrawal of the instrument from the trocar, the spacing allows for the instrument shaft to remain in a fluid tight seal with diaphragm seal 34 as the instrument tip is withdrawn through the opening for forward valve 22. In this way, the spacing aspects facilitate improved fluid pressure management during endoscopic surgeries.

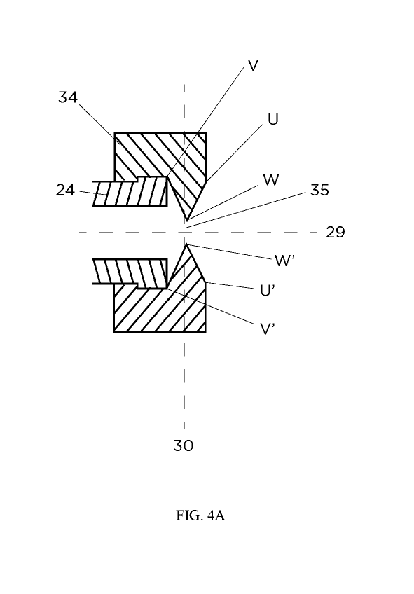

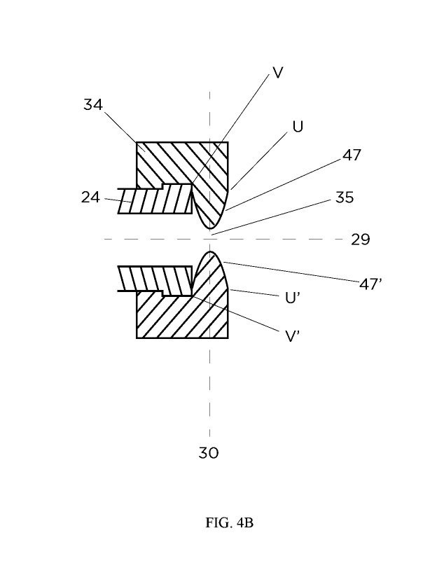

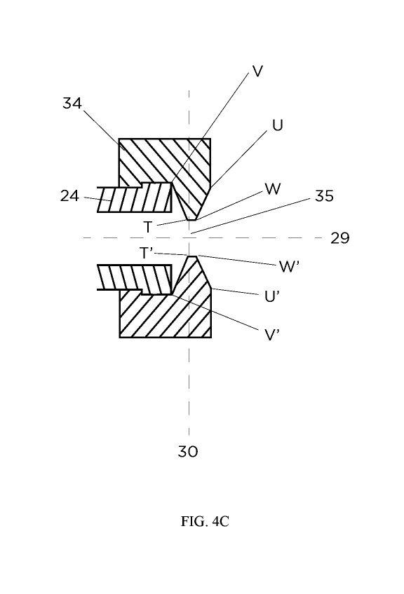

The opposing tapered portions of diaphragm seal 34 promote a fluid tight seal around an instrument shaft. FIG. 4A provides an enlarged area of region C of FIG. 3, and FIGS. 4B-4C show alternative arrangements. While the views provide additional geometric information, other arrangements are also contemplated. In some embodiments, points U and V (and likewise U′ and V′) represent the points where diaphragm seal 34 begins to taper inward. Although appearing as distinct lines in the cross-sectional drawing figure, in actuality segment VW together with V′W′ represent a surface of diaphragm seal 34 which generally faces a forward insert 27 of the trocar (see FIG. 1). Likewise, segment UW together with U′W′ together represent a generally rear-insert facing surface of diaphragm seal 34, which opposes the forward-insert facing surface. Accordingly, each opposing surface of diaphragm seal 34 tapers inward toward the center of diaphragm seal 34 relative to two distinct axes. That is, as shown in this cross-sectional view, the opposing surfaces represented by segments VW and UW are not parallel, and both segments taper inward toward the center of diaphragm seal 34 relative to one axis which can be considered central axis 29, and relative to a second axis 30 that is perpendicular to axis 29. Likewise, segments V′W′ and U′W′ in this figure are not parallel, and both segments taper inward toward the center of diaphragm seal 34 relative to the same two axes. In some embodiments, on both opposing surfaces, the slope of the taper ranges from about 5 degrees to about 30 degrees from an outer periphery of the diaphragm seal toward the opening 35 (or, more specifically, toward the intersection of central axis 29 and perpendicular axis 30 as depicted in FIG. 4A). In some embodiments, the taper is from about 15 degrees to about 25 degrees (about 15 degrees is preferable), and the opposing surfaces are tapered symmetrically to enhance efficiencies of inserting and withdrawing of an instrument.

Referring still to FIGS. 4A-4C, in some embodiments the distance from where the taper begins (i.e., point V and V′) to the center of opening 35 will vary depending on the type of instrument used, the diameter of the shaft of the instrument, and the needs of the particular surgical patient.

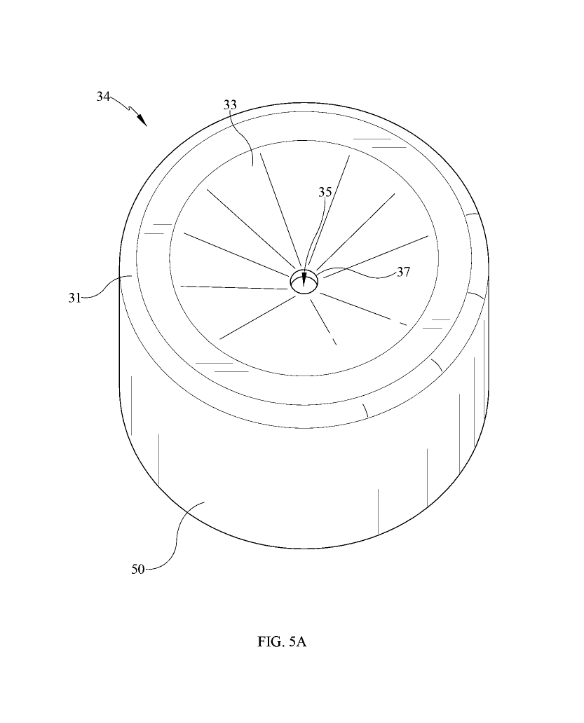

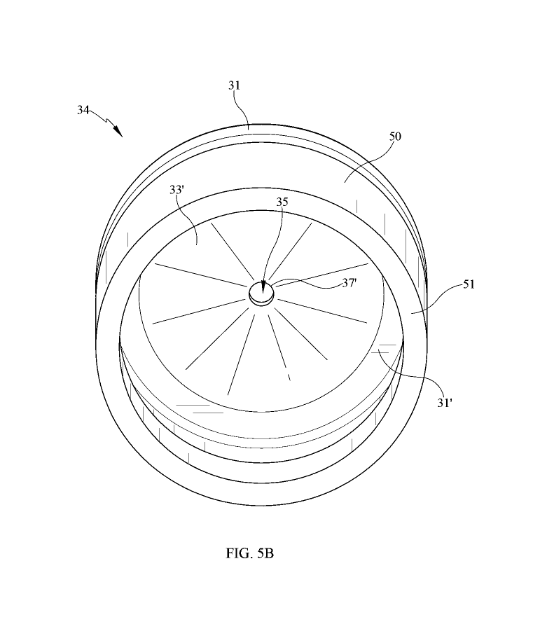

Referring back to FIG. 1, end cap 24 and diaphragm seal 34 are illustrated in relation to housing 12 and also identifying with a reference numeral the forward insert 27 which enters a patient's body compartment. Now with reference to FIGS. 5A and 5B, to facilitate providing a fluid tight seal, each of one or more diaphragm seals 34 of trocar 10 has a dual taper, on opposing sides. In FIG. 5A, a surface of diaphragm seal 34 is shown, which comprises a semi-cylindrical tapered portion 33 descending from an outer edge 31 toward the center of the diaphragm seal to a circular rim 37. Thus, in some embodiments, rim 37 forms part of a boundary for an opening 35 extending through the full thickness of seal 34. In some embodiments, opening 35 has a diameter of from about 1 mm to about 10 mm. Though various figures illustrate rim 37 as circular in geometry, other geometries are contemplated within the presently described embodiments.

A dual taper is provided by opposing tapered portions of diaphragm seal 34. FIG. 5B shows an opposing surface of diaphragm seal 34 from that shown in FIG. 5A. It is also comprised of a tapered portion 33′ descending from outer edge 31′ toward the center of the diaphragm seal to form a circular rim 37′ which also serves as part of a boundary for the aforementioned opening 35. Each surface represented by the tapered portions, 33, 33′ tapers in an opposing direction, thus providing the dual taper of diaphragm seal 34 and effectively bringing the respective tapered portions closer to the other as they approach opening 35. Accordingly, in some embodiments, the general shape of each tapered portion can be considered as conical, or as a truncated cone, terminating at opening 35. As shown in FIG. 4B, the cross-sectional surface areas of the tapered portions surrounding opening 35 are curvilinear surfaces 47, 47′ respectively, with that area being of sufficient thickness so that opening 35 maintains adequate contact with the instrument shaft to establish the desired fluid tight seal.

Alternatively, as shown in FIG. 4C, the cross-sectional surface areas of the tapered portions surrounding opening 35 create a truncated cone—represented by segments VT-TW-WU and V′T′-T′W′-W′U′, respectively—again providing area of sufficient thickness so that opening 35 maintains adequate contact with the instrument shaft to establish and maintain a fluid tight seal. Such contact and the resultant sealing force are established and maintained even while surgeons move the instrument through various ranges of motion, resulting in fluctuating and intermittent compressive forces and release of those forces over the surface area that defines opening 35.

Having sufficient surface area of material around opening 35 to remain in contact with an instrument shaft helps form and maintain a satisfactory fluid tight seal. For added versatility, opening 35 of diaphragm seal 34 can be made of an appropriate size and shape to accommodate instrument shafts of various diameters to form a fluid tight seal therebetween with an abutting surface of an instrument shaft (such as instrument shaft 5 depicted in FIG. 2) and rim 37, 37′ of the diaphragm seal 34. Generally, it is beneficial for the diameter of the opening 35 of diaphragm seal 34 to be slightly smaller than the diameter of the intended instrument shaft. The tapered portion of the diaphragm seal 34 not only serves to form and augment a fluid tight seal, but also provides flexibility with respect to the range of instruments having various shaft diameters that are suitable for use with trocars as described herein. Additionally, the surfaces of forward-facing tapered portion 33′ function not only as part of the seal interface, but also serve to redirect fluid away from opening 35 by causing the flow path of the fluid to revert toward the inner chamber 26 of the housing 12 through deflection.

In use, as a surgeon passes an instrument through a trocar, the instrument advances axially and is withdrawn axially in a generally linear direction along axis 29. In so doing, the instrument shaft 5 slideably passes through and interfaces with the opening 35 of diaphragm seal 34. In practice, a surgeon generally must be able to move the instrument, including shaft 5 (FIG. 2), with significant range of motion and along numerous axes relative to axis 29. For example, an instrument tip may be formed with a blunt edge spiral thread to help advance the tip through the body compartment wall as the surgeon rotates the body of the trocar, and additional maneuvering is frequently needed once the surgical site is accessed. In conventional designs, such movement along multiple axes often produces or exacerbates leakage. Accordingly, the tapered portions on the rear insert-facing side of the diaphragm seal 34 and the forward insert-facing side thereof, facilitate multi-axis movement of the instrument without disrupting the engagement required for a the fluid tight seal. Additionally, as seen in FIG. 5B and the cross-sectional figures of the diaphragm seal region, in some embodiments the taper associated with the forward insert-facing wall 33′ will—when an instrument is being withdrawn from trocar 10—tend to urge fluid to rebound off of the surface of the instrument shaft 5 and away from the seal in a direction toward the forward insert 27, thereby promoting the recycling of fluid into the body compartment, thus increasing the effectiveness of insufflating the body compartment.

According to present embodiments, the spatial relationships and distances between diaphragm seal 34 and valve 22 are such that the instrument tip does not impinge upon or enter that valve until the diaphragm seal 34 has engaged an abutting surface of the instrument shaft 5 and established a fluid tight seal therebetween. With further reference to FIG. 2 and FIG. 3, in some embodiments, the distance between valves 22, 23 is such that when withdrawing an instrument, forward valve 22, e.g., a multi-leaflet valve, sufficiently closes and forms a relatively fluid tight seal between opposing surfaces of the valve leaflets before the tip of the instrument begins to pass back through rear valve 23 and, in turn, out of trocar 10 through diaphragm seal 34. In some embodiments, each diaphragm seal(s) 34 is arranged so that the interface formed therebetween with instrument shaft 5 withstands pressures at and beyond what is typically exerted by means of insufflation, e.g., for laparoscopy an insufflation pressure on the order of about 15 mm Hg, and for arthroscopy involving a joint capsule an insufflation pressure on the order of about 70 mm Hg.

Thus, features of trocars described herein facilitate a desired pressure to be maintained within the trocar and, in turn, within the body compartment where surgery is performed, whereas with conventional designs, this often is not the case. With some conventional trocars, the distance between the diaphragm seal and the multi-leaflet valve is so small that it is prone to leakage either when the first instrument is inserted, or when that one is withdrawn and subsequent instruments are inserted then withdrawn. In contrast, according to present embodiments, the opening-to-opening distance between diaphragm seal 34 and forward valve 22 exceeds the length of most instrument tips. By way of non-limiting example, in some embodiments this distance is no less than about 30 mm (3 cm), but other distances are contemplated.

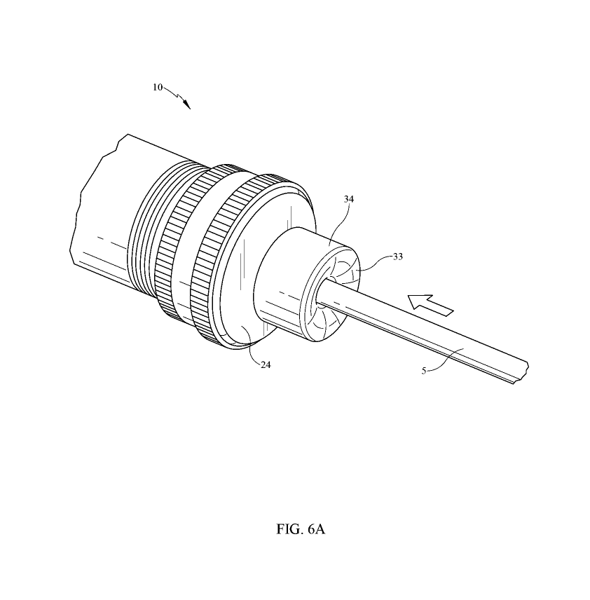

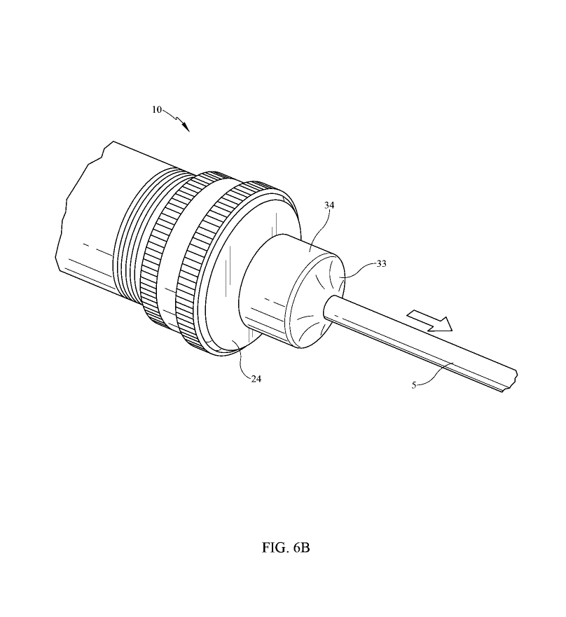

FIGS. 6A and 6B demonstrate the resilient nature of diaphragm seal 34 according to present embodiments. In FIG. 6A, the forward-pointing arrow signifies insertion of instrument shaft moving toward the forward insert 27 of earlier figures. In response to this forward advancement, tapered portion 33 is seen as somewhat concave due to the force placed upon this surface as a result of the seal created between diaphragm 34 and instrument tip 5 as the instrument advances. Conversely, FIG. 6B's rear-pointing arrow (away from rear insert 25 of earlier figures) is suggestive of withdrawing the instrument, in which the tapered portion 33 bows outward by the created seal.

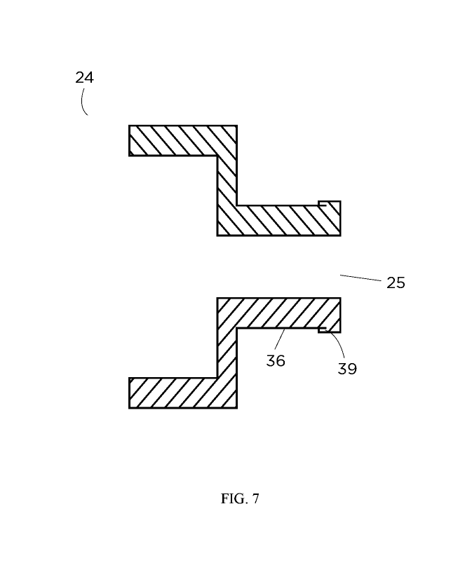

In some embodiments, diaphragm seal 34 fits over an end cap joined to the trocar. FIG. 7 provides a cross-sectional view of end cap 24, which in some embodiments is cylindrical. End cap 24 includes a diaphragm seat region 36 for accommodating a diaphragm seal 34. With respect to diaphragm seal 34, rear-facing tapered portion 33 meets rim 31 at the point where the taper begins to angularly slope inwardly and radially toward opening 35. In turn, rim 31 also meets a diaphragm collar 50, which in some embodiments extends at approximately 90 degrees relative to the rim in a forward orientation when diaphragm seal 34 is attached to trocar 10. The collar 50 terminates at a forward periphery 51, which defines an axially aligned open end to enable diaphragm seal 34 to engage with end cap 24. Preferably, this open end, the outer portions of which are defined by forward periphery 51, thus defines an area appropriately sized for diaphragm seal 34 to fit tightly over end cap 24. The geometry of diaphragm seal 34 is complementary to that of end cap 24, and/or a diaphragm seat region 36 of the end cap which is discussed below in connection with FIG. 7. Accordingly, if the geometry of end cap 24 (or, diaphragm seat region 36) is cylindrical, the geometry of the forward periphery 51 is likewise cylindrical.

In general, the fit between diaphragm seal 34 and end cap 24 is sufficiently secure for the diaphragm seal to remain in place even during forceful manipulations of the instrument associated with surgical techniques, yet able to be manually removed from end cap 24 if desired. One way to accomplish this is to have the diameter of the forward periphery of 51 diaphragm seal 34 equal to or slightly less than the outer diameter of end cap 24 or the diaphragm seat region 36, such that the flexible material of diaphragm seal 34 allows it to be stretched and force fit over this outer diameter.

As well, other structures can be employed to hold diaphragm seal 34 to end cap 24. FIG. 7 illustrates end cap 24 having diaphragm seat region 36, which includes a lip 39 that protrudes slightly from the outer diameter of diaphragm seat region 36. Accordingly, lip 39 provides a capturing ledge to secure the positioning of diaphragm seal 34 so it does not slide off when the trocar is under pressure. End cap 24 is thus arranged to be positioned at an open end of housing 12 proximal to rear insert 25 shown in other figures. In some embodiments, end cap 24 has mating threads (not visible in the figure, but these would be formed along the inner diameter of the end cap). The threads are adapted to mate with corresponding external threads 42 located on housing 12, as FIG. 8 illustrates. FIG. 8, a cross-sectional view of a trocar housing taken axially, further shows a valve seat region 36 for accommodating valves 22, 23, as previously discussed in proximal relation to forward insert 27 of trocar 10. Accordingly, for reusable versions of trocar 10, end cap 24 is removable by turning the end cap in a direction opposite that which was used to secure it to the diaphragm seat region 36. This provides a way for end cap 24 to be cleaned between uses, or for cleaning or replacement of diaphragm seal 34 and valves 22, 23.

It will be understood that the embodiments described herein are not limited in their application to the details of the teachings and descriptions set forth, or as illustrated in the accompanying figures. Rather, it will be understood that the present embodiments and alternatives, as described and claimed herein, are capable of being practiced or carried out in various ways. Also, it is to be understood that words and phrases used herein are for the purpose of description and should not be regarded as limiting. The use herein of such words and phrases as “such as,” “comprising,” “e.g.,” “containing,” or “having” and variations of those words is meant to encompass the items listed thereafter, and equivalents of those, as well as additional items. The use of “including” (or, “include,” etc.) should be interpreted as “including but not limited to.”

Accordingly, the foregoing descriptions of several embodiments and alternatives are meant to illustrate, rather than to serve as limits on the scope of what has been disclosed herein. The descriptions herein are not intended to be exhaustive, nor are they meant to limit the understanding of the embodiments to the precise forms disclosed. It will be understood by those having ordinary skill in the art that modifications and variations of these embodiments are reasonably possible in light of the above teachings and descriptions.

What is claimed is:

1. A trocar for use with a surgical instrument, comprising:
a trocar housing with an inner chamber, a rear insert and a forward insert;
at least one tapered diaphragm seal, having two non-parallel, opposing surfaces that taper from an outer edge of the diaphragm seal to form an opening;
a forward valve and a rear valve, each having a valve opening, wherein the rear valve opening is closer to the diaphragm seal opening than is the forward valve opening, and the distance between the rear valve and the forward valve is substantially statically maintained.

2. The trocar of claim 1, wherein the distance from the diaphragm seal opening to the rear valve opening is at least about 2 cm, and the distance from the diaphragm seal opening to the forward valve opening is at least about 7 cm.

3. The trocar of claim 1, further comprising a spacer sleeve positioned between the forward valve and the rear valve for maintaining the distance between them.

4. The trocar of claim 1, further comprising forward and rear grooves formed in the trocar housing providing a secure seating arrangement for said forward and rear valves and for maintaining the distance between them.

5. The trocar of claim 1, wherein at least one diaphragm seal is external to the trocar housing and configured for placement over a diaphragm seat region.

6. The trocar of claim 1, wherein at least one diaphragm seal is positioned within the trocar housing.

7. The trocar of claim 1, wherein the opposing surfaces taper toward the diaphragm seal opening at an angle of about 5 to about 30 degrees.

8. The trocar of claim 7, wherein the opposing surfaces taper toward the opening at an angle of about 15 to about 25 degrees.

9. The trocar of claim 1, wherein the cross-sectional surface areas of the tapered portions at the opening are curvilinear.

10. The trocar of claim 1, wherein the cross-sectional surface areas of the tapered portions at the opening are a truncated cone.

11. A diaphragm seal for a trocar, comprising two non-parallel, opposing surfaces that taper from an outer edge of the diaphragm seal to form an opening, wherein the opposing surfaces taper toward the opening at an angle of about 5 to about 30 degrees, and wherein the diaphragm seal is configured to be secured to an end cap of the trocar.

12. The diaphragm seal of claim 11, wherein the opposing surfaces taper toward the diaphragm seal opening at an angle of about 5 to about 30 degrees.

13. The diaphragm seal of claim 12, wherein the opposing surfaces taper toward the opening at an angle of about 15 to about 25 degrees.

14. The diaphragm seal of claim 11, wherein the cross-sectional surface areas of the tapered portions at the opening are curvilinear.

15. The diaphragm seal of claim 11, wherein the cross-sectional surface areas of the tapered portions at the opening are a truncated cone.
